(19) United States
(12) Patent Application Publication
(10) Pub. No.: US 2026/0365787 A1
(43) Pub. Date: Nov. 19, 2026

(54) PURIFIED BIOMASS BASED COMPOSITIONS COMPRISING ETHYLENE GLYCOL AND UE THEREOF TO PRODUCE A POLYESTER

(71) Applicant: TOPSOE A/S, Kgs. Lyngby (DK)

(72) Inventors: Torkil Ottesen HANSEN, Charlottenlund (DK); Farhad VARZANDEH, Nærum (DK); Lars Skov JENSEN, Lejre (DK); Esben TAARNING, Frederiksberg (DK); Lars Saaby PEDERSEN, Farum (DK)

(73) Assignee: TOPSOE A/S, Kgs. Lyngby (DK)

(21) Appl. No.: 19/136,517

(22) PCT Filed: Dec. 21, 2023
(86) PCT No.: PCT/EP2023/087238

§ 371 Date: Jun. 6, 2025

(30) Foreign Application Priority Data
Dec. 22, 2022 (EP) 22216010.3

(51) Int. Cl. C08G 63/183; C07C 29/78; C07C 29/80

(52) U.S. Cl. CPC C08G 63/183; C07C 29/78; C07C 29/80

(57) ABSTRACT

A method of purifying a biomass based composition including ethylene glycol, the method including: (a) subjecting the biomass based composition to at least one distillation step to provide a distillation product, wherein the concentration of ethylene glycol in the distillation product is greater than in the biomass based composition; and (b) subjecting the distillation product to at least one melt crystallisation step to form crystals and a mother liquor, such that the crystals provide a purified biomass based composition, wherein the concentration of ethylene glycol in the purified biomass based composition is greater than in the distillation product.

### FIELD

The present invention relates to biomass based compositions comprising ethylene glycol, and to methods and uses relating thereto. The present invention further relates to methods of producing a polyester using biomass based compositions.

### BACKGROUND

Ethylene glycol is an organic polyol having the IUPAC name ethane-1,2-diol. At present, ethylene glycol is most commonly obtained from fossil fuel sources. A typical method for producing fossil based ethylene glycol uses ethylene produced from oil. The ethylene is oxidised in the presence of a catalyst to form ethylene oxide, which is then hydrolysed to form ethylene glycol.

In view of a changing environmental and economic climate, there is a desire to use ethylene glycol obtained from renewable sources, such as biomass, e.g. sugars. For example, WO2016001169A1 discloses a method of producing ethylene glycol from sugars, involving pyrolysis of sugar to form mixed C1-C3 oxygenates (e.g. formaldehyde, glycolaldehyde, glyoxal, acetol, and pyruvaldehyde), which are hydrogenated in the presence of a catalyst to form a crude ethylene glycol product.

One of the main industrial uses of ethylene glycol is as a raw material for the production of polyesters. Polyesters have a wide range of applications, such as for the production of packaging articles (e.g. bottles), textiles, and electronic goods. A polyester of particular industrial importance is polyethylene terephthalate (PET). A number of patent applications disclose methods of purifying a biomass based, crude ethylene glycol product to produce high purity ethylene glycol (>99 wt % EG) mentioned to be suitable for producing PET. For example, WO 2015/150520 and WO 2022/223867 disclose a purification method involving distillation to obtain a high purity ethylene glycol. In CN 106866371A a multi-stage crystallisation is disclosed as a purification method for producing ethylene glycol having a purity of 98.5%-99.9% from a crude ethylene glycol product obtained from e.g. HTHP oxalate hydrogenation. None of the patent applications address the problems associated with impurities nor do they measure UV transmittance or APHA colour of the purified ethylene glycol composition. Neither is actual PET synthesised and thus, there is no measure of characteristics of the PET, such as the CIELAB colour.

Packaging articles are often required to meet strict technical specifications in relation to colour properties. There is also an expectation in the art for ethylene glycol compositions used to produce packaging articles to have certain technical properties in order to produce packaging articles that meet the requisite technical specifications. For example, a widely accepted view in the art is that ethylene glycol compositions require a high UV transmittance in order to produce polyester packaging articles that meet the requisite technical specifications. This may for example be seen in Zhang et al., “Identification of impurities affecting commercial ethylene glycol UV transmittance”, J Chromatogr A 904 (2000) 87-97.

The article refers to the issue of low UV transmittance of ethylene glycol rendering it unsuitable as a raw material for making polyesters. The article goes on to identify the presence of some major UV absorbing impurities which they suggest to remove (without stating how) to obtain “polymer grade” ethylene glycol. The introduction mentions “Ethylene glycol that is used to make polyesters should be of exceptionally high purity and must meet a special UV transmittance specification, requiring that ethylene glycol have UV transmittances of at least 75%, 95%, 100% at 220, 275 and 350 nm, respectively. It is believed that low UV transmittance at these wavelengths indicates the presence of undesirable impurities that reduce the resulting polyester quality.”

This assumption about UV transmittance is also clearly deducible from the fact that producers of polyester grade monoethylene glycol specify a minimum UV transmittance at 350, 275 and 220 nm in their product specifications. For example LyondellBasell (US sales specification for monoethylene glycol, polyester grade, material number 5017), MEGlobal (sales specification for monoethylene glycol, polyester grade, specified material 000101232907, revision 1 Jan. 2019, and sales specification for commercial polyester grade, specified material 000101205133, revision 1 Jan. 2019) and SABIC (technical data for monoethylene glycol, bulk, revision 20220825) all specify minimum UV transmittances of 98% at 350 nm, 90-94% at 275 nm, and 70% at 220 nm.

Furthermore, a number of patent applications relating to biobased polyester grade ethylene glycol use UV transmittance at 350, 275 and 220 nm as a goal to be achieved, see e.g. WO 2015/028156, WO 2018/089600, WO 2018/089605, CN101525424A and CN 104418997A. For example, CN101525424A mentions that the biobased ethylene glycol suitable for preparing PET must have a transmittance in the wavelength region of 190~350 nm of more than 50%.

It would be desirable to provide biomass based compositions comprising ethylene glycol that can be used to produce polyesters and packaging articles satisfying the requisite or desired technical specifications for polyesters and packaging articles. It also would be desirable to provide economical and industrially feasible methods of producing such biomass based compositions and polyesters.

### SUMMARY

According to an aspect of the present invention, there is provided a method of purifying a biomass based composition comprising ethylene glycol, the method comprising: (a) subjecting the biomass based composition to at least one distillation step to provide a distillation product, wherein the concentration of ethylene glycol in the distillation product is greater than in the biomass based composition; and (b) subjecting the distillation product to at least one melt crystallisation step to form crystals and a mother liquor, such that the crystals provide a purified biomass based composition, wherein the concentration of ethylene glycol in the purified biomass based composition is greater than in the distillation product.

It is to be understood that when referring to “at least one melt crystallisation step” this implies that the invention encompasses conducting a single melt crystallisation step but it also encompasses conducting several melt crystallisation steps, e.g. two, three or four melt crystallisation steps.

In one aspect, the distillation product comprises ethylene glycol in an amount of no less than 85 wt. %, such as no less than 90 wt. %, such as no less than 95 wt. %, based on the weight of the distillation product.

In one aspect, the distillation product comprises ethylene glycol in an amount of no greater than 99.95 wt. %, such as no greater than 99.9 wt. %, based on the weight of the distillation product. The purified biomass based composition has an absolute upper limit of ethylene glycol of 100 wt. % based on the weight of the purified biomass based composition.

In one aspect, the purified biomass based composition comprises ethylene glycol in an amount of no less than 98 wt. %, such as no less than 99 wt. %, such as no less than 99.25 wt. %, such as no less than 99.5 wt. %, such as no less than 99.75 wt. %, such as no less than 99.9 wt. %, based on the weight of the purified biomass based composition.

In one aspect, the at least one distillation step is a vacuum distillation step.

In one aspect, the at least one distillation step is performed at a pressure of from 100 mbar to 750 mbar, such as from 150 mbar to 300 mbar.

In one aspect, the at least one distillation step comprises a first distillation step comprising feeding the biomass based composition to a continuous distillation unit to provide a first bottom fraction and at least one first distillate fraction wherein one of the first bottom fraction and the at least one first distillate fraction is an ethylene glycol enriched fraction. This ethylene glycol enriched fraction may be the distillation product.

In one aspect, the at least one distillation step comprises at least one further distillation step comprising feeding the ethylene glycol enriched fraction from the preceding distillation step to a continuous distillation unit to provide a further bottom fraction and at least one further distillate fraction wherein one of the further bottom fraction and the at least one further distillate fraction is an ethylene glycol enriched fraction. This ethylene glycol enriched fraction may be the distillation product.

In one aspect, the biomass based composition is obtained by thermolytic fragmentation of a sugar.

In one aspect, the or each melt crystallisation step comprises cooling the distillation product using a heat exchanger, wherein the temperature of the heat exchanger is no greater than the freezing temperature of ethylene glycol.

In one aspect, the temperature of the heat exchanger is no greater than −12.9 °C, such as no greater than −15 °C, such as no greater than −18 °C, such as no greater than −20 °C, and/or the temperature of the heat exchanger is no less than −40 °C, such as no less than −35 °C, such as no less than −30 °C, such as no less than −25 °C.

In one aspect, step (b) comprises subjecting the distillation product to the at least one melt crystallisation step to form the crystals and the mother liquor, the crystals being present in an amount of at least 50 wt. %, such as no greater than 90 wt. %, such as from 60 wt. % to 70 wt. %, based on the weight of the crystals and the mother liquor.

In one aspect, the or each melt crystallisation step is a suspension melt crystallisation step. In one aspect, the or each melt crystallisation step comprises heating the crystals to partially melt the crystals to form purified crystals and a residual liquid, wherein the concentration of ethylene glycol in the purified crystals is greater than in the crystals.

In one aspect, the or each melt crystallisation step comprises heating the crystals to partially melt the crystals to form the purified crystals and the residual liquid, such that from 10 wt. % to 50 wt. % of the crystals are melted to form the residual liquid, based on the weight of the crystals.

In one aspect, the at least one distillation step comprises at least partially removing from the biomass based composition one or more of: water, propylene glycol, and 1,2-butanediol.

In one aspect, the distillation product comprises one or more of: 1,2-pentanediol, 1,2-cyclopentanediol, 1,2-hexanediol, and 1,2-cyclohexanediol.

In one aspect, the at least one melt crystallisation step comprises at least partially removing from the distillation product one or more of: 1,2-pentanediol, 1,2-cyclopentanediol, 1,2-hexanediol, and 1,2-cyclohexanediol.

In one aspect, step (b) comprises subjecting the distillation product to the at least one melt crystallisation step to form the crystals and the mother liquor, wherein the concentration of one or more of 1,2-pentanediol, 1,2-cyclopentanediol, 1,2-hexanediol, and 1,2-cyclohexanediol is greater in the mother liquor than in the crystals.

In one aspect, the purified biomass based composition has a UV transmittance at 275 nm determined in accordance with ASTM method E2193-16 of less than 40%, such as no greater than 35%, such as no greater than 30%, such as no greater than 20%.

In one aspect, the purified biomass based composition is characterised by an APHA colour value determined according to ASTM D1209-05 of no greater than 5 mg/L PtCo.

In one aspect, the purified biomass based composition is characterised by an APHA colour value after heating determined according to ASTM D1209-05 of no greater than 20 mg/L PtCo.

According to another aspect of the present invention, there is provided a purified biomass based composition obtained by the method of purifying a biomass based composition according to an above aspect of the present invention, the purified biomass based composition being characterised by an APHA colour after heating determined according to ASTM D1209-05 of no greater than 20 mg/L PtCo.

According to another aspect of the present invention, there is provided a purified biomass based composition obtained by the method of purifying a biomass based composition according to an above aspect of the present invention, the purified biomass based composition being characterised by an APHA colour after heating determined according to ASTM D1209-05 of no greater than 15 mg/L PtCo.

According to another aspect of the present invention, there is provided a use of a purified biomass based composition obtained by the method of purifying a biomass based composition according to an above aspect of the present invention to produce a polyester where the polyester is characterised by one or more of the following CIELAB colour space values determined according to ASTM D6290-19: L* of no less than 65; a* of from −4 to 4; and b* of from −4 to 4.

According to another aspect of the present invention, there is provided a method of producing a polyester, the method comprising contacting the purified biomass based composition obtained by the method of purifying a biomass based composition according to an above aspect of the present invention with at least one reagent to form the polyester characterised by one or more of the following CIELAB colour space values determined according to ASTM D6290-19: L* of no less than 65; a* of from −4 to 4; and b* of from −4 to 4.

According to another aspect of the present invention, there is provided a polyester obtained by a method of producing a polyester according to an above aspect of the present invention, the polyester being characterised by one or more of the following CIELAB colour space values determined according to ASTM D6290-19: L* of no less than 65; a* of from −4 to 4; and b* of from −4 to 4.

In one aspect, the polyester is characterised by one or more of the following CIELAB colour space parameter values determined according to ASTM D6290-19: L* of no less than 85; a* of from −2 to 2; and b* of from −2 to 2. In one aspect, the polyester is characterised by the CIELAB colour space parameter value b* determined according to ASTM D6290-19 of from −4 to 4, preferably-2 to 2. In one aspect, the polyester is characterised by the CIELAB colour space parameter value a* determined according to ASTM D6290-19 of from −4 to 4, preferably-2 to 2. In one aspect, the polyester is characterised by the CIELAB colour space parameter value L* determined according to ASTM D6290-19 of no less than 65, preferably no less than 85.

According to another aspect of the present invention, there is provided a packaging article or a preform formed from the polyester according to an above aspect of the present invention.

In any one of the above aspects of the present invention, the polyester may comprise polyethylene terephthalate.

The features of any aspect of the present invention may be combined with any feature or features of any other aspect of the present invention.

### DETAILED DESCRIPTION

### Distillation and Melt Crystallisation

As discussed herein, in an aspect of the present invention there is provided a method of purifying a biomass based composition comprising ethylene glycol, the method comprising: (a) subjecting the biomass based composition to at least one distillation step to provide a distillation product, wherein the concentration of ethylene glycol in the distillation product is greater than in the biomass based composition; and (b) subjecting the distillation product to at least one melt crystallisation step to form crystals and a mother liquor, such that the crystals provide a purified biomass based composition, wherein the concentration of ethylene glycol in the purified biomass based composition is greater than in the distillation product.

Ethylene glycol (monoethylene glycol) has many applications, including as antifreeze and as a monomer for making polyesters. To be used as a monomer for making polyesters, a minimum purity of ethylene glycol may be desired or required. For example, to be used as a monomer for making bottle grade polyesters (e.g. bottle grade polyethylene terephthalate), high purity ethylene glycol may be desired or required. If the ethylene glycol is not of a sufficient purity, the ethylene glycol may not meet product specifications and/or may produce a polyester that has undesirable characteristics (e.g. an undesirable colour profile, such as a yellow colour).

Distillation has been used to purify fossil based compositions comprising ethylene glycol compositions. We have found that distillation as a sole technique cannot be used to purify biomass based compositions comprising ethylene glycol to the desired level with economic feasibility. While distillation may be used to adequately remove impurities from fossil based compositions comprising ethylene glycol, biomass based compositions comprising ethylene glycol have a different impurity profile and impurities therein are difficult to remove using distillation alone. The challenge of providing an economically feasible purification method as regards biomass based compositions comprising ethylene glycol has been a major obstacle for delivering a more sustainable source material for the preparation of polyesters.

We have identified that a combination of distillation and melt crystallisation can be used to purify biomass based compositions comprising ethylene glycol to the desired level in an economically feasible way. More specifically, we have found that distillation can be used to efficiently prepare a semi-pure composition (i.e. the distillation product), and that melt crystallisation can be used to purify the semi-pure composition to provide a high purity composition (i.e. the purified biomass based composition) enriched in ethylene glycol. A significant energy saving can be achieved by combining the distillation and melt crystallisation techniques. We have identified that this combination of techniques can be significantly more energy efficient that using either technique alone.

### Purification

Herein, “purifying” or “purified” or “purification” can be considered as enrichment of ethylene glycol in a composition by removal of other components from the composition. “Other components” may include impurities, i.e. components present in small concentration.

### Distillation

The method of purifying the biomass based composition comprises step (a) subjecting the biomass based composition to at least one distillation step to provide a distillation product, wherein the concentration of ethylene glycol in the distillation product is greater than in the biomass based composition.

Herein, the “distillation product” can be a bottom fraction or a distillate fraction. Herein, a “bottom fraction” (or “bottoms”) can be considered as a liquid fraction that collects at the bottom of the distillation column during distillation. Those skilled in the art will appreciate that the bottom fraction comprises components which are less volatile than the components of the distillate fraction. Herein, a “distillate fraction” can be considered a fraction of vapour or liquid condensed from vapour, or a mix of vapour and liquid, that is removed from the distillation column at any point above the bottom of the distillation column during distillation. At least one distillate fraction is collected during distillation, although multiple distillate fractions can be collected from the distillation column simultaneously. Hence, a “liquid side draw” is a distillate fraction, which is not collected from the top of the distillation column.

In one aspect, the biomass based composition that is subjected to the at least one distillation step is aqueous.

In one aspect, the biomass based composition comprises one or more of: water, propylene glycol, ethylene glycol, and 1,2-butanediol.

In one aspect, the biomass based composition comprises propylene glycol in an amount of no less than 0.01 wt. %, based on the weight of the biomass based composition. In one aspect, the biomass based composition comprises propylene glycol in an amount of no less than 0.1 wt. %, based on the weight of the biomass based composition. In one aspect, the biomass based composition comprises propylene glycol in an amount of no less than 0.2 wt. %, based on the weight of the biomass based composition. In one aspect, the biomass based composition comprises propylene glycol in an amount of no less than 0.5 wt. %, based on the weight of the biomass based composition. In one aspect, the biomass based composition comprises propylene glycol in an amount of no less than 1 wt. %, based on the weight of the biomass based composition.

In one aspect, the biomass based composition comprises propylene glycol in an amount of no greater than 10 wt. %, based on the weight of the biomass based composition. In one aspect, the biomass based composition comprises propylene glycol in an amount of no greater than 8 wt. %, based on the weight of the biomass based composition. In one aspect, the biomass based composition comprises propylene glycol in an amount of no greater than 6 wt. %, based on the weight of the biomass based composition. In one aspect, the biomass based composition comprises propylene glycol in an amount of no greater than 5 wt. %, based on the weight of the biomass based composition. In one aspect, the biomass based composition comprises propylene glycol in an amount of no greater than 3 wt. %, based on the weight of the biomass based composition.

In one aspect, the biomass based composition comprises 1,2-butanediol in an amount of no less than 0.01 wt. %, based on the weight of the biomass based composition. In one aspect, the biomass based composition comprises 1,2-butanediol in an amount of no less than 0.1 wt. %, based on the weight of the biomass based composition. In one aspect, the biomass based composition comprises 1,2-butanediol in an amount of no less than 0.2 wt. %, based on the weight of the biomass based composition. In one aspect, the biomass based composition comprises 1,2-butanediol in an amount of no less than 0.5 wt. %, based on the weight of the biomass based composition. In one aspect, the biomass based composition comprises 1,2-butanediol in an amount of no less than 1 wt. %, based on the weight of the biomass based composition.

In one aspect, the biomass based composition comprises 1,2-butanediol in an amount of no greater than 10 wt. %, based on the weight of the biomass based composition. In one aspect, the biomass based composition comprises 1,2-butanediol in an amount of no greater than 8 wt. %, based on the weight of the biomass based composition. In one aspect, the biomass based composition comprises 1,2-butanediol in an amount of no greater than 6 wt. %, based on the weight of the biomass based composition. In one aspect, the biomass based composition comprises 1,2-butanediol in an amount of no greater than 5 wt. %, based on the weight of the biomass based composition. In one aspect, the biomass based composition comprises 1,2-butanediol in an amount of no greater than 3 wt. %, based on the weight of the biomass based composition.

In one aspect, the at least one distillation step comprises at least partially removing from the biomass based composition one or more of: water, propylene glycol, and 1,2-butanediol.

In one aspect, the distillation product comprises ethylene glycol in an amount of no greater than 99.95 wt. %, such as no greater than 99.9 wt. %, based on the weight of the distillation product.

In one aspect, the distillation product comprises one or more of: 1,2-pentanediol, 1,2-cyclopentanediol, 1,2-hexanediol, and 1,2-cycolhexanediol.

In one aspect, the at least one distillation step is a vacuum distillation step.

In one aspect, the at least one distillation step is performed at a pressure of from 100 mbar to 750 mbar. In one aspect, the at least one distillation step is performed at a pressure of from 150 mbar to 300 mbar.

In one aspect, the at least one distillation step comprises a first distillation step comprising feeding the biomass based composition to a continuous distillation unit to provide a first bottom fraction and at least one first distillate fraction wherein one of the first bottom fraction and the at least one first distillate fraction is an ethylene glycol enriched fraction.

This ethylene glycol enriched fraction may be the distillation product.

In one aspect, the at least one distillation step comprises at least one further distillation step comprising feeding the ethylene glycol enriched fraction from the preceding distillation step to a continuous distillation unit to provide a further bottom fraction and at least one further distillate fraction wherein one of the further bottom fraction and the at least one further distillate fraction is an ethylene glycol enriched fraction. This ethylene glycol enriched fraction may be the distillation product.

Herein, “enriched” is relative to the composition on which the distillation was performed. For example, the ethylene glycol enriched fraction from the third distillation step is enriched in ethylene glycol relative to the ethylene glycol enriched fraction from the second distillation step.

In one aspect, the ethylene glycol enriched fraction from the first distillation step is also enriched in propylene glycol. This fraction is referred to as an ethylene glycol enriched and propylene glycol enriched fraction from the first distillation step.

In one aspect, the at least one distillation step comprises a second distillation step comprising feeding the ethylene glycol enriched fraction from the first distillation step to a continuous distillation unit to provide a second bottom fraction and at least one second distillate fraction wherein one of the second bottom fraction and the at least one second distillate fraction is an ethylene glycol enriched fraction. This ethylene glycol enriched fraction may be the distillation product.

In one aspect, the at least one distillation step comprises a second distillation step comprising feeding the ethylene glycol enriched and propylene glycol enriched fraction from the first distillation step to a continuous distillation unit to provide a second bottom fraction and at least one second distillate fraction wherein one of the second bottom fraction and the at least one second distillate fraction is an ethylene glycol enriched fraction. This ethylene glycol enriched fraction may be the distillation product.

In one aspect, the at least one distillation step comprises a third distillation step comprising feeding the ethylene glycol enriched fraction from the second distillation step to a continuous distillation unit to provide a third bottom fraction and at least one third distillate fraction wherein one of the third bottom fraction and the at least one third distillate fraction is an ethylene glycol enriched fraction. This ethylene glycol enriched fraction may be the distillation product.

In one aspect, the at least one distillation step comprises a fourth distillation step comprising feeding the ethylene glycol enriched fraction from the third distillation step to a continuous distillation unit to provide a fourth bottom fraction and at least one fourth distillate fraction wherein one of the fourth bottom fraction and the at least one fourth distillate fraction is an ethylene glycol enriched fraction. This ethylene glycol enriched fraction may be the distillation product.

In one aspect, at least 80 wt. %, such as at least 85 wt. %, such as at least 90 wt. %, such as at least 92 wt. %, such as at least 95 wt. %, such as at least 97.5 wt. %, such as at least 99 wt. %, such as at least 99.5 wt. % of non-ethylene glycol components (i.e. all components which are not ethylene glycol) are removed from the biomass based composition in the at least one distillation step (in this instance, the complete distillation sequence), based on the weight of the biomass based composition.

For a given distillation, the split of the feed between top and bottom is controlled by the feed rate, the reflux ratio, the energy input to the reboiler, the preheating of the feed, the input of cooling media to the reflux and distillate condenser, the pressure and the separation power of the column, as well as the vapor-liquid equilibrium for the components in the feed.

Components of higher volatility than ethylene glycol may be concentrated and removed as distillate, and a concentrated ethylene glycol product may be collected as bottom fraction. Alternatively, components of lower volatility than ethylene glycol may be concentrated and removed as bottom fraction, and a concentrated ethylene glycol product may be collected as distillate.

It is within the knowledge of the skilled person within distillation technology to design a distillation step providing either an ethylene glycol enriched bottom fraction or an ethylene glycol enriched distillate fraction.

### Biomass Based Composition

In one aspect, the biomass based composition has a ¹⁴C content above 0.5 parts per trillion of the total carbon content as determined by ASTM D6866-22.

In one aspect, the biomass based composition is obtained by thermolytic fragmentation and subsequent hydrogenation of biomass. Alternatively, the biomass based composition may be obtained by hydrogenolysis of biomass. Hydrogenolysis is a chemical reaction analogous to hydrolysis in which hydrogen plays a role similar to that of water. In hydrogenolysis, a bond, typically in an organic molecule, is broken, with the simultaneous addition of a hydrogen atom to each of the resulting molecular fragments. In one aspect, the biomass comprises one or more of lignocellulose, lignin, sewage sludge, lipids, proteins and carbohydrates.

Thermolytic fragmentation (or pyrolysis) of biomass describes processes where a biomass feedstock is subjected to a thermal treatment resulting in a partial breakdown of its constituents to produce a pyrolysed product. Thermolytic fragmentation of glucose and hydrogenation of the resulting pyrolysate is known according to e.g. Schandel et al., ChemSusChem, 2020, 13, 688-692, U.S. Pat. No. 9,926,247, and WO 2017/216311.

Biomass includes all types of biogenic materials, i.e. materials which are made from the fixation of atmospheric CO₂ within recent time (i.e. the last century). This includes lignocellulose, lignin, sewage sludge, lipids, proteins and carbohydrates.

The group of carbohydrates comprises polysaccharides, oligosaccharides and sugars. The group of polysaccharides comprises long polymers of sugars and includes cellulose, hemicellulose and starch. The group of oligosaccharides comprises short polymers of sugars (4-10 monosaccharide units). The group of sugars comprises trisaccharides, disaccharides and monosaccharides. The group of trisaccharides includes maltotriose. The group of disaccharides includes sucrose, maltose, lactose and cellobiose. The group of monosaccharides comprises all monosaccharides in the groups of trioses, tetroses, pentoses and hexoses and preferred monosaccharides are pentoses and hexoses, more preferred are glucose, fructose, mannose, galactose, xylose and arabinose or mixtures of these. Most preferred is glucose as a monosaccharide feedstock. The monosaccharide feedstock can contain up to 5% by weight of di- and tri-saccharides relative to the monosaccharide and still be considered as a monosaccharide feedstock.

In one aspect, the biomass based composition is obtained by thermolytic fragmentation and subsequent hydrogenation of carbohydrates. In one aspect, the biomass based composition is obtained by thermolytic fragmentation and subsequent hydrogenation of polysaccharides, oligosaccharides, sugars and mixtures thereof. In one aspect, the biomass based composition is obtained by thermolytic fragmentation and subsequent hydrogenation of sugars. In one aspect, the biomass based composition is obtained by thermolytic fragmentation and subsequent hydrogenation of polysaccharides, oligosaccharides, trisaccharides, disaccharides, monosaccharides and mixtures thereof. In one aspect, the biomass based composition is obtained by thermolytic fragmentation and subsequent hydrogenation of disaccharides, monosaccharides and mixtures thereof. In one aspect, the biomass based composition is obtained by thermolytic fragmentation and subsequent hydrogenation of at least disaccharides. In one aspect, the biomass based composition is obtained by thermolytic fragmentation and subsequent hydrogenation of at least monosaccharides.

Thermolytic fragmentation describes processes where heat is applied to the biomass feedstock (typically 300-700 °C) for a certain amount of time to bring about its transformation into a pyrolysed product (or thermolytic fragmentation product). Thermolytic fragmentation does not include conditions or processes where substantial combustion, or gasification into permanent gases, of the feedstock is achieved. The heat can be applied by allowing a small amount of the feedstock to combust by introduction of oxygen or an oxidant, or heat can be applied externally e.g. by contact with a hot surface, gas, liquid or solid body to transfer heat into the feedstock. Thermolytic fragmentation can be carried out in different reactors such as bubbling fluidised bed reactors, circulating fluidised bed reactors, ablative reactors, rotating cone reactors, micro-pyrolyser units, etc. The heat can be applied over long time (slow pyrolysis, >5 minutes), medium time (conventional pyrolysis, 30-300 seconds) and short times (fast pyrolysis such as less than 30 seconds and typically around 0.5-2 seconds).

The thermolytic fragmentation duration and temperatures influence the composition of the pyrolysed product.

Monosaccharide pyrolysis describes the transformation of a monosaccharide feedstock (i.e. a monosaccharide feedstock which does not contain substantial amounts of lignocellulose, lignin, lipids, cellulose, hemicellulose, starch, proteins, oligosaccharides, trisaccharides and disaccharides) by pyrolysis into a pyrolysed product. Monosaccharide pyrolysis which is carried out using a monosaccharide feedstock containing less than 15 wt. % water, based on the weight of the monosaccharide feedstock, is referred to as ‘dry monosaccharide pyrolysis’. Monosaccharide pyrolysis which is carried out on a monosaccharide feedstock containing more than 15 wt. % water, based on the weight of the monosaccharide feedstock, is referred to as ‘wet sugar pyrolysis’. The goal for wet monosaccharide pyrolysis may be to transform an aqueous monosaccharide feedstock into glycolaldehyde (2-hydroxyacetaldehyde) together with the formation of other light oxygenates (pyruvaldehyde, acetol, formaldehyde and glyoxal) and with minimal formation of other products.

The monosaccharide feedstock for wet monosaccharide pyrolysis is an aqueous solution of monosaccharides containing more than 15 wt. %, such as more than 20 wt. %, more than 30 wt. %, more than 40 wt. %, more than 50 wt. %, more than 60 wt. %, more than 70 wt. %, or more than 80 wt. % water, based on the weight of the monosaccharide feedstock. An example of a monosaccharide feedstock for wet monosaccharide pyrolysis is a feedstock comprising 64 wt. % glucose, 1 wt. % maltose (disaccharide) and 35 wt. % water. Another example of a monosaccharide feedstock for wet monosaccharide pyrolysis is a feedstock comprising 32 wt. % glucose, 31 wt. % fructose, 1.5 wt. % sucrose and 35.5 wt. % water.

A monosaccharide feedstock for dry monosaccharide pyrolysis comprises less than 15 wt. % water, based on the weight of the monosaccharide feedstock. An example of a monosaccharide feedstock for dry monosaccharide pyrolysis is glucose monohydrate (91 wt. % monosaccharide and 9 wt. % water).

After thermolytic fragmentation of the feedstock, the pyrolysis product which has been formed may be subjected to hydrogenation.

Hydrogenation refers to a chemical reaction between molecular hydrogen and another compound or element, optionally in the presence of a catalytic material and optionally in the presence of a solvent. In the present process, glycolaldehyde may be formed from the thermolytic fragmentation of the biomass, such as the pyrolysis of sugars, and in this aspect hydrogenation of the pyrolysis product transforms glycolaldehyde into ethylene glycol, optionally together with transformation of glyoxal into ethylene glycol, pyruvaldehyde and acetol into propylene glycol and formaldehyde into methanol and with minimal formation of other products. The resulting composition may be referred to as a biomass based composition.

Suitable hydrogenation catalysts comprises an active material selected from the group consisting of ruthenium, rhenium, rhodium, iridium, palladium, platinum, copper and nickel; or mixtures thereof, on a support. The support material is normally made of an inert material. Suitable support materials are carbon, silica, alumina, titania, and zirconia; or mixtures thereof.

In one aspect, the pyrolysis product is subjected to a gas phase hydrogenation in the presence of hydrogen and a hydrogenation catalyst. When the hydrogenation is a gas phase hydrogenation, the hydrogenation may be conducted at a temperature in the range of from 200 °C to 250 °C and a hydrogen partial pressure in the range of from 0.5 bar to 5 bar.

In one aspect, the pyrolysis product is subjected to a liquid phase hydrogenation in the presence of hydrogen and a hydrogenation catalyst. When the hydrogenation is a liquid phase hydrogenation, the hydrogenation may be conducted at a temperature in the range of from 60 °C to 120 °C and a hydrogen partial pressure in the range of from 20 bar to 200 bar. When the hydrogenation is a liquid phase hydrogenation, the partial pressure of hydrogen is the partial pressure in the gas phase above, or interspersed with, the hydrogenation fluid, which is proportional to the concentration of hydrogen in the liquid phase.

In one aspect, the hydrogenation is conducted in the presence of a solvent selected from the group consisting of water, methanol, ethanol, ethylene glycol and propylene glycol; and mixtures thereof.

Prior to the hydrogenation, the pyrolysis product may be subjected to operations such as condensation and/or separation.

In one aspect, the biomass based composition has a total aldehyde concentration of no less than 100 ppm. In one aspect, the biomass based composition has a total aldehyde concentration of no less than 200 ppm. In one aspect, the biomass based composition has a total aldehyde concentration of no less than 300 ppm. In one aspect, the biomass based composition has a total aldehyde concentration of no less than 500 ppm. In one aspect, the biomass based composition has a total aldehyde concentration of no less than 1000 ppm. “ppm” may be based on the weight of the biomass based composition.

In one aspect, the biomass based composition has a total aldehyde concentration of no greater than 1000000 ppm. In one aspect, the biomass based composition has a total aldehyde concentration of no greater than 100000 ppm. In one aspect, the biomass based composition has a total aldehyde concentration of no greater than 10000 ppm. In one aspect, the biomass based composition has a total aldehyde concentration of no greater than 8000 ppm. In one aspect, the biomass based composition has a total aldehyde concentration of no greater than 5000 ppm. “ppm” may be based on the weight of the biomass based composition.

In one aspect, the biomass based composition has a total aldehyde concentration of from 100 ppm to 1000000 ppm. In one aspect, the biomass based composition has a total aldehyde concentration of from 200 ppm to 100000 ppm. In one aspect, the biomass based composition has a total aldehyde concentration of from 300 ppm to 10000 ppm. In one aspect, the biomass based composition has a total aldehyde concentration of from 500 ppm to 8000 ppm. In one aspect, the biomass based composition has a total aldehyde concentration of from 1000 ppm to 5000 ppm. “ppm” may be based on the weight of the biomass based composition.

### Melt Crystallisation

The method of purifying the biomass based composition comprises subjecting the distillation product to at least one melt crystallisation step to form crystals and a mother liquor, such that the crystals provide a purified biomass based composition, wherein the concentration of ethylene glycol in the purified biomass based composition is greater than in the distillation product.

As will be understood by those skilled in the art, subjecting a composition to “melt crystallisation” can mean lowering the temperature of the composition to induce formation of crystals. For example, a component of the composition may crystallise as the temperature of the composition is lowered to (or beyond) the freezing point of said component. Those skilled in the art will understand that melt crystallisation is distinct from solution crystallisation. For example, melt crystallisation is temperature driven and typically performed in the absence of a crystallisation solvent, whereas solution crystallisation is solubility (e.g. supersaturation) driven and involves separation of crystals from a crystallisation solvent.

As will be understood by those skilled in the art, a purpose of the at least one melt crystallisation step is to remove impurities from the distillation product, so as to increase the concentration of ethylene glycol in the purified biomass based composition relative to in the distillation product.

In one aspect, the or each melt crystallisation step comprises at least partially (or completely) removing the mother liquor from the crystals.

In one aspect, the or each melt crystallisation step comprises cooling the distillation product using a heat exchanger, wherein temperature of the heat exchanger is no greater than the freezing temperature of ethylene glycol.

In one aspect, the temperature of the heat exchanger is no greater than −12.9 °C. In one aspect, the temperature of the heat exchanger is no greater than −15 °C. In one aspect, the temperature of the heat exchanger is no greater than −18 °C. In one aspect, the temperature of the heat exchanger is no greater than −20 °C.

In one aspect, the temperature of the heat exchanger is no less than −40 °C. In one aspect, the temperature of the heat exchanger is no less than −35 °C. In one aspect, the temperature of the heat exchanger is no less than −30 °C. In one aspect, the temperature of the heat exchanger is no less than −25 °C.

In one aspect, step (b) comprises subjecting the distillation product to the at least one melt crystallisation step to form the crystals and the mother liquor, the crystals being present in an amount of at least 50 wt. %, based on the weight of the crystals and the mother liquor. In one aspect, step (b) comprises subjecting the distillation product to the at least one melt crystallisation step to form the crystals and the mother liquor, the crystals being present in an amount of no greater than 90 wt. %, based on the weight of the crystals and the mother liquor. In one aspect, step (b) comprises subjecting the distillation product to the at least one melt crystallisation step to form the crystals and the mother liquor, the crystals being present in an amount of from 60 wt. % to 70 wt. %, based on the weight of the crystals and the mother liquor.

In one aspect, the or each melt crystallisation step is a suspension melt crystallisation step.

In one aspect, the or each melt crystallisation step comprises heating the crystals to partially melt the crystals to form purified crystals and a residual liquid, wherein the concentration of ethylene glycol in the purified crystals is greater than in the crystals. “Heating the crystals to partially melt the crystals to form purified crystals and a residual liquid” may be referred to as a “sweating step”.

In one aspect, the or each melt crystallisation step comprises at least partially (or completely) removing the residual liquid from the purified crystals.

In one aspect, the or each melt crystallisation step comprises heating the crystals to partially melt the crystals to form the purified crystals and the residual liquid, such that from 10 wt. % to 50 wt. %, such as from 10 wt. % to 40 wt. % of the crystals are melted to form the residual liquid, based on the weight of the crystals.

In one aspect, the at least one melt crystallisation step comprises at least partially removing from the distillation product one or more of: 1,2-pentanediol, 1,2-cyclopentanediol, 1,2-hexanediol, and 1,2-cyclohexanediol.

In one aspect, step (b) comprises subjecting the distillation product to the at least one melt crystallisation step to form the crystals and the mother liquor, wherein the concentration of one or more of 1,2-pentanediol, 1,2-cyclopentanediol, 1,2-hexanediol, and 1,2-cyclohexanediol is greater in the mother liquor than in the crystals.

In some aspects the biomass based composition may in addition be subjected to a step of contacting the biomass based composition with a solid acid catalyst and/or an aldehyde removal resin.

### Purified Biomass Based Composition

The purified biomass based composition is provided by the crystals from step (b) of the method of purifying a biomass based composition. The purified biomass based composition may be considered as the biomass based composition that is to be used for polyester production. As discussed herein, the crystals may be subjected to one or more further steps before being used for polymer production.

In one aspect, the purified biomass based composition has a total aldehyde concentration of no greater than 50 ppm. In one aspect, the purified biomass based composition has a total aldehyde concentration of no greater than 20 ppm. In one aspect, the purified biomass based composition has a total aldehyde concentration of no greater than 18 ppm. In one aspect, the purified biomass based composition has a total aldehyde concentration of no greater than 15 ppm. In one aspect, the purified biomass based composition has a total aldehyde concentration of no greater than 10 ppm. “ppm” may be based on the weight of the biomass based composition.

In one aspect, the purified biomass based composition has a total aldehyde concentration of no less than 1 ppm. In one aspect, the purified biomass based composition has a total aldehyde concentration of no less than 2 ppm. In one aspect, the purified biomass based composition has a total aldehyde concentration of no less than 5 ppm. In one aspect, the purified biomass based composition has a total aldehyde concentration of no less than 10 ppm. “ppm” may be based on the weight of the biomass based composition.

In one aspect, the purified biomass based composition is characterised by an APHA colour value (determined according to ASTM D1209-05) of no greater than 10 mg/L PtCo. In one aspect, the purified biomass based composition is characterised by an APHA colour value (determined according to ASTM D1209-05) of no greater than 9 mg/L PtCo. In one aspect, the purified biomass based composition is characterised by an APHA colour value (determined according to ASTM D1209-05) of no greater than 8 mg/L PtCo. In one aspect, the purified biomass based composition is characterised by an APHA colour value (determined according to ASTM D1209-05) of no greater than 7 mg/L PtCo. In one aspect, the purified biomass based composition is characterised by an APHA colour value (determined according to ASTM D1209-05) of no greater than 6 mg/L PtCo. In one aspect, the purified biomass based composition is characterised by an APHA colour value (determined according to ASTM D1209-05) of no greater than 5 mg/L PtCo.

The American Public Health Association (APHA) colour scale, also known as the Hazen scale or Platinum-Cobalt (PtCo) scale, is a measure of the colour of liquid chemicals. The scale is used to evaluate the quality of chemicals and other substances, and it is a good measure of the level of impurities affecting colour in e.g. an biomass based composition comprising ethylene glycol.

Herein, the APHA colour value is determined according to ASTM D1209-05 at a temperature of from 20 °C to 25 °C (room temperature) and at atmospheric pressure.

The APHA colour value may also be used to assess the thermal stability of a liquid chemical by testing of “APHA colour after heating”. In the present context, this is done by conducting a heating step, where the liquid chemical is heated to 200 degrees for an extended period of time. In the present context, 2 to 4 hours is suitable. After cooling to room temperature, the APHA colour value is determined. The APHA colour value may be used as an indicator of the thermal stability of the liquid. A high APHA colour value could indicate that the substance has degraded or reacted to form coloured impurities during heating.

Where nothing else is stated herein, the APHA colour refers to testing of the purified biomass based composition without performing the heating step. If a heating step has been conducted, the reference will be “APHA colour after heating”.

We have surprisingly found that an ethylene glycol composition may be polymer grade even though it does not meet the industrial specifications for UV transmittance at 220 nm, 275 nm and 350 nm. We found that an APHA colour before heating of below 5 mg/L PtCo and an APHA colour after heating of below 20 mg/L PtCo are good indicators as to whether an ethylene glycol composition may be polymer grade/suitable for producing colourless PET. It is even better if APHA colour after heating is below 15 mg/L.

Accordingly, the thermal stability/the APHA colour after heating may be used as a measure for the suitability of a purified biomass based composition comprising ethylene glycol as an ethylene glycol reactant in synthesis of PET. In an embodiment according to the invention, the purified biomass based composition comprising ethylene glycol which is considered suitable as reactant in synthesis of PET has an APHA colour after heating of below 20 mg/L PtCo. In an embodiment, the purified biomass based composition comprising ethylene glycol according to the invention considered suitable as reactant in synthesis of PET has an APHA colour after heating of below 15 mg/L PtCo.

In one aspect, the purified biomass based composition is characterised by an APHA colour value (determined according to ASTM D1209-05) of more than 0 mg/L PtCo, and this may be considered as a lower limit for any of the upper limits mentioned herein.

In one aspect, the purified biomass based composition comprises ethylene glycol in an amount of no less than 98 wt. %, based on the weight of the purified biomass based composition. In one aspect, the purified biomass based composition comprises ethylene glycol in an amount of no less than 99 wt. %, based on the weight of the purified biomass based composition. In one aspect, the purified biomass based composition comprises ethylene glycol in an amount of no less than 99.25 wt. %, based on the weight of the purified biomass based composition. In one aspect, the purified biomass based composition comprises ethylene glycol in an amount of no less than 99.5 wt. %, based on the weight of the purified biomass based composition.

In one aspect, the purified biomass based composition comprises ethylene glycol in an amount of no greater than 99.75 wt. %, based on the weight of the purified biomass based composition. In one aspect, the purified biomass based composition comprises ethylene glycol in an amount of no greater than 99.9 wt. %, based on the weight of the purified biomass based composition

In one aspect, the purified biomass based composition has a UV transmittance at 275 nm (determined in accordance with ASTM method E2193-16) of less than 40%.

UV transmittance is used as a simple measure for the absence of low concentration impurities in ethylene glycol having adverse effect on the colour in a PET obtained therefrom, i.e. higher transmittance means less impurities” When removing low concentration impurities from ethylene glycol, the purity of ethylene glycol becomes higher as a consequence of removing the impurities. Hitherto, a widely accepted understanding in the art of the present invention is that ethylene glycol compositions require a high UV transmittance in order to be suitable for producing polyesters having an acceptable polymer quality. This is particularly applicable in the context of bottle manufacture, e.g. polyethylene terephthalate bottle manufacture. Purification of biomass based ethylene glycol to achieve a high UV transmittance can be expensive and complex, if possible at all (See e.g. WO 2015/028156, WO 2018/089600 and WO 2018/089605). This inability to deliver biomass based ethylene glycol meeting the specifications on UV transmittance in an economical way has been a major obstacle for delivering a more sustainable source material for the preparation of polyesters. We have found, surprisingly, that low UV transmittance biomass based ethylene glycol can be used to produce satisfactory “bottle grade” polyesters even though the biomass based ethylene glycol did not meet the specifications on UV transmittance.

Herein, UV transmittance at 275 nm is determined in accordance with ASTM method E2193-16 at a temperature of from 20 °C to 25 °C; and at atmospheric pressure.

In one aspect, the purified biomass based composition has a UV transmittance at 275 nm (determined in accordance with ASTM method E2193-16) of no greater than 38%. In one aspect, the purified biomass based composition has a UV transmittance at 275 nm (determined in accordance with ASTM method E2193-16) of no greater than 35%. In one aspect, the purified biomass based composition has a UV transmittance at 275 nm (determined in accordance with ASTM method E2193-16) of no greater than 32%. In one aspect, the purified biomass based composition has a UV transmittance at 275 nm (determined in accordance with ASTM method E2193-16) of no greater than 30%. In one aspect, the purified biomass based composition has a UV transmittance at 275 nm (determined in accordance with ASTM method E2193-16) of no greater than 28%. In one aspect, the purified biomass based composition has a UV transmittance at 275 nm of no greater than 25%. In one aspect, the purified biomass based composition has a UV transmittance at 275 nm (determined in accordance with ASTM method E2193-16) of no greater than 22%. In one aspect, the purified biomass based composition has a UV transmittance at 275 nm (determined in accordance with ASTM method E2193-16) of no greater than 20%.

In one aspect, the purified biomass based composition has a UV transmittance at 275 nm (determined in accordance with ASTM method E2193-16) of no less than 0%. In one aspect, the purified biomass based composition has a UV transmittance at 275 nm (determined in accordance with ASTM method E2193-16) of no less than 1%. In one aspect, the purified biomass based composition has a UV transmittance at 275 nm (determined in accordance with ASTM method E2193-16) of no less than 2%. In one aspect, the purified biomass based composition has a UV transmittance at 275 nm (determined in accordance with ASTM method E2193-16) of no less than 5%. In one aspect, the purified biomass based composition has a UV transmittance at 275 nm (determined in accordance with ASTM method E2193-16) of no less than 8%. In one aspect, the purified biomass based composition has a UV transmittance at 275 nm (determined in accordance with ASTM method E2193-16) of no less than 10%. In one aspect, the purified biomass based composition has a UV transmittance at 275 nm (determined in accordance with ASTM method E2193-16) of no less than 12%. In one aspect, the purified biomass based composition has a UV transmittance at 275 nm (determined in accordance with ASTM method E2193-16) of no less than 15%.

In one aspect, the purified biomass based composition has a UV transmittance at 275 nm (determined in accordance with ASTM method E2193-16) of between 0% and 40%. In one aspect, the purified biomass based composition has a UV transmittance at 275 nm (determined in accordance with ASTM method E2193-16) of from 1% to 38%. In one aspect, the purified biomass based composition has a UV transmittance at 275 nm (determined in accordance with ASTM method E2193-16) of from 2% to 38%. In one aspect, the purified biomass based composition has a UV transmittance at 275 nm (determined in accordance with ASTM method E2193-16) of from 5% to 35%. In one aspect, the purified biomass based composition has a UV transmittance at 275 nm (determined in accordance with ASTM method E2193-16) of from 10% to 35%. In one aspect, the purified biomass based composition has a UV transmittance at 275 nm (determined in accordance with ASTM method E2193-16) of from 15% to 35%. In one aspect, the purified biomass based composition has a UV transmittance at 275 nm (determined in accordance with ASTM method E2193-16) of from 20% to 35%.

In an embodiment according to the invention, the purified biomass based composition comprising ethylene glycol which is considered suitable as reactant in synthesis of PET has an APHA colour after heating below 20 mg/L PtCo. In an embodiment, the purified biomass based composition comprising ethylene glycol according to the invention considered suitable as reactant in synthesis of PET has an APHA colour after heating below 15 mg/L PtCo.

### Composition

According to another aspect of the present invention, there is provided a purified biomass based composition obtained by the method of purifying the biomass based composition according to an aspect of the present invention.

According to another aspect of the present invention, there is provided a purified biomass based composition obtained by the method of purifying a biomass based composition according to an above aspect of the present invention, the purified biomass based composition being characterised by an APHA colour after heating of below 20 mg/L PtCo as determined according to ASTM D1209-05 and an UV transmittance at 275 nm of below 40% as determined by ASTM method E2193-16.

According to another aspect of the present invention, there is provided a purified biomass based composition obtained by the method of purifying a biomass based composition according to an above aspect of the present invention, the purified biomass based composition being characterised by an APHA colour after heating of below 15 mg/L PtCo (as determined according to ASTM D1209-05) and an UV transmittance at 275 nm of below 40% (as determined by ASTM method E2193-16).

### Use

According to another aspect of the present invention, there is provided a use of the purified biomass based composition obtained by the method of purifying a biomass based composition according to an aspect of the present invention to produce a polyester.

The polyester may comprise polyethylene terephthalate.

### Polyester Production

According to another aspect of the present invention, there is provided a method of producing a polyester, the method comprising contacting the purified biomass based composition obtained by the method of purifying a biomass based composition according to an aspect of the present invention with at least one reagent to form a polyester.

In one aspect, the contacting the purified biomass based composition obtained by the method of purifying the biomass based composition with at least one reagent comprises: (I) reacting ethylene glycol with the at least one reagent to provide monomers; and (II) polymerising the monomers to provide the polyester. However, for the avoidance of doubt, the polyester may be polymerised by any method suitable to obtain the desired polyester properties.

In one aspect, the at least one reagent comprises one or more of a diacid, a diester, and an acid anhydride.

In one aspect, the diacid is a terephthalic compound, an isophthalic compound, or a mixture thereof.

In one aspect, the diacid is renewably sourced. For example, the diacid may be formed from a synthetic route from a biomass-derived starting material, such as furfural.

In one aspect, the terephthalic compound is selected from the group consisting of terephthalic acid, dimethyl terephthalate, or a combination thereof.

In one aspect, the isophthalic compound is selected from isophthalic acid, dimethyl isophthalate, or a combination thereof.

In one aspect, the diacid is selected from naphthalenedicarboxylic acid, cyclohexanedicarboxylic acid, cyclohexanediacetic acid, succinic acid, glutaric acid, furandicarboxylic acid, adipic acid, azelaic acid, sebacic acid, and combinations thereof.

In one aspect, the monomers comprise bis(2-hydroxyethyl) terephthalate monomers.

The polyester may comprise polyethylene terephthalate. In one aspect, the contacting the purified biomass based composition obtained by the method of purifying the biomass based composition with at least one reagent comprises: (1) reacting ethylene glycol with one or more of a diacid and a diester to provide bis(2-hydroxyethyl) terephthalate monomers; and

(II) polymerising the monomers to provide the polyester comprising polyethylene terephthalate. In one aspect, step (I) of the method of producing a polyester is performed at a temperature of from 230 °C to 260 °C. In one aspect, step (II) of the method of producing a polyester is performed at a temperature of from 270 °C to 300 °C.

In one aspect, step (II) of the method of producing a polyester is performed in the presence of a catalyst. In one aspect, the catalyst is a heterogeneous catalyst. In one aspect, the catalyst is an antimony-containing catalyst or a platinum-containing catalyst, or a titanium-containing catalyst, or an aluminium-containing catalyst, or a germanium-containing catalyst. In one aspect, the catalyst is antimony (III) oxide. In one aspect, step (II) of the method of producing a polyester is performed in the presence of phosphorous compounds added as stabilizer.

The polyester is considered to be polyethylene terephthalate (PET) when the ethylene glycol fraction of the diols in the polyester is above 90% and when the terephthalic acid fraction of the diacids in the polyester is above 90%.

In one aspect, the step (I) of reacting ethylene glycol with the at least one reagent to provide monomers may be performed in the presence of minor amounts of other diols. In the present context a minor amount is preferably below 40 mole % of the total molar amount of diols.

In one aspect the other diols are selected from the group comprising diethylene glycol, 1,3-propanediol, 1,4-butanediol, 1,5-pentanediol, 2-methyl-1,4-pentanediol, 3-methyl2,4-pentanediol, trimethyl-1,3-pentanediol, 2-ethyl-1,3-hexanediol, 2,2-diethyl-1,3propanediol, 1,3-hexanediol, 1,4-di(hydroxyethoxy)benzene, 2,2-bis(4hydroxycyclohexyl) propane, 2,4-dihydroxy-1,1,3,3-tetramethylcyclobutane, 2,2-bis(3hydroxyethoxyphenyl) propane, 2,2-bis(4-hydroxypropoxyphenyl) propane, and cyclohexanedimethanol, or mixtures thereof.

The conditions of polymerisation may produce polymer grades having variable molecular weights. Polymers with different molecular weights are traditionally described by their intrinsic viscosity (IV) values, as measured by ASTM D792.

In one aspect, a solid-state polymerisation (SSP) reaction is further performed in the polyester at a temperature from 180 to 230 °C under inert or low-pressure atmosphere for a given time to yield the desired values of IV. In one aspect, the IV of the polyester after SSP is at least 0.65 dL/g. In one aspect, the IV of the polyester after SSP is at least 0.75 dL/g. In one aspect, the IV of the polyester after SSP is at least 0.85 dl/g. In one aspect, the IV of the polyester after SSP is at least 1.0 dL/g.

### Polyester

According to another aspect of the present invention, there is provided a polyester obtained by a method producing a polyester according to an aspect of the present invention.

In one aspect, polyester composition disclosed herein may include an additive in addition to the polyester. An additive may include but is not limited to colorant, ultraviolet (UV) stabilizer, antioxidant, filler, gas barrier additive, plasticizer, nucleating agent, thermal stabilizer, chain extender, and a combination thereof.

An additive may be incorporated into polyester compositions described herein using known methods. For example, an additive may be introduced before, during, or after the polymerisation step. An additive may also be compounded with the polyester in subsequent steps of processing or transformation.

In one aspect, polyesters according to one or more embodiments and articles thereof can be recycled by using conventional recycling methods under recycling operational conditions known by a person having ordinary skill in the art, such as mechanical and chemical recycling. In one or more embodiments, the packaging article produced with the polyester disclosed herein is mechanically recycled in the form of chips or granules. Therefore, the resulting chips and granules will typically retain some biogenic carbon content as measurable by ¹⁴C methods and ASTM D6866. The resulting chips or granules might be reprocessed into the same or different polyester-based packaging articles using the processing and manufacturing techniques described herein. The reprocessing may occur simultaneously, but not exclusively, with PET from conventional petrochemical sources (from fossil fuel or non bio-based sources) or chips and granules obtained by recycling conventional polyester.

In one aspect, the polyester is characterised by an L* CIELAB colour space parameter value (determined in accordance with ASTM method D6290-19) of no less than 65. In one aspect, the polyester is characterised by an L* CIELAB colour space parameter value (determined in accordance with ASTM method D6290-19) of no less than 70. In one aspect, the polyester is characterised by an L* CIELAB colour space parameter value (determined in accordance with ASTM method D6290-19) of no less than 75. In one aspect, the polyester is characterised by an L* CIELAB colour space parameter value (determined in accordance with ASTM method

D6290-19) of no less than 80. In one aspect, the polyester is characterised by an L* CIELAB colour space parameter value (determined in accordance with ASTM method D6290-19) of no less than 85. In one aspect, the polyester is characterised by an L* CIELAB colour space parameter value (determined in accordance with ASTM method D6290-19) of no less than 90.

Herein, the CIELAB colour space parameter values are determined in accordance with ASTM D6290-19 at a temperature of from 20 °C to 25 °C (room temperature) and at atmospheric pressure.

In one aspect, the polyester is characterised by an a* CIELAB colour space parameter value (determined in accordance with ASTM method D6290-19) of from −4 to 4. In one aspect, the polyester is characterised by an a* CIELAB colour space parameter value (determined in accordance with ASTM method D6290-19) of from −3 to 3. In one aspect, the polyester is characterised by an a* CIELAB colour space parameter value (determined in accordance with ASTM method D6290-19) of from −2 to 2.

In one aspect, the polyester is characterised by a b* CIELAB colour space parameter value (determined in accordance with ASTM method D6290-19) of from −4 to 4. In one aspect, the polyester is characterised by a b* CIELAB colour space parameter value (determined in accordance with ASTM method D6290-19) of from −3 to 3. In one aspect, the polyester is characterised by a b* CIELAB colour space parameter value (determined in accordance with ASTM method D6290-19) of from −2 to 2.

The polyester may comprise polyethylene terephthalate.

### Packaging Article or Preform

According to another aspect of the present invention, there is provided a packaging article or a preform formed from the polyester according to an aspect of the present invention.

Polyesters in accordance with the present disclosure may be formulated for a number of polymer articles and products. Polyester articles may include but are not limited to containers, flasks, bottles, vessels, containers, caps, carpet, clothing, fabrics, strapping, rope, fiberfill, construction materials, furniture, medical applications, film, sheet, lamination, protective packaging, electrical encapsulation, solenoids, smart meters, photovoltaic parts, solar junction boxes, automotive parts, wiper arm and gear housings, headlamp retainers, engine covers, connector housings, industrial fibers, 3D printing filament, thermoformed articles and the like.

As above, an application for polyester in accordance with one or more embodiments is a film. In particular, a polyester film application may be a monoaxially oriented film, a biaxially oriented film, a multilayered film with other polymeric materials, a blow-molded film, and an article, or an extrusion coating. A blow-molded article may be extrusion blown, stretch blown, or injection blown.

In one or more embodiments, the polyesters can be used for producing polyester fibers from melt-spinning and/or drawing. Polyester fibers may include but is not limited to draw texturized yarn, partially oriented yarn, polyester staple fibers, fully drawn yarn, spin draw yarn, and polyester mesh.

Manufacturing processes may include but are not limited to injection molding, stretch blow molding, lamination, extrusion, thermoforming, melt-spinning, and 3D printing.

In one aspect, the packaging article is a container. In one aspect, the polyester container may be used but is not limited to packaging food, cosmetics, soft drinks, water, alcoholic beverages, cosmetics, pharmaceuticals and edible oils.

In one aspect, the packaging article is a bottle.

The polyester may comprise polyethylene terephthalate.

### Total Aldehyde Concentration

The total aldehyde concentration may be determined by any suitable technique as will be known to those skilled in the art. For example, in one aspect, the total aldehyde concentration is determined using ASTM method E2313-20.

### Ethylene Glycol Concentration

The concentration of ethylene glycol may be determined by any suitable technique as will be known to those skilled in the art. In one aspect, the concentration of ethylene glycol is determined using flame ionization detection (FID) gas chromatography (GC), referred to herein as “GC-FID”.

Any aspect of the present disclosure may be defined in relation to any of the other aspects of the present disclosure. For example, one aspect of the present disclosure may include any of the features of any other aspect of the present disclosure. For example, the features of one aspect of the present disclosure may be as defined in relation to the features of any other aspect of the present disclosure.

### EXAMPLES

### Example 1: GC Analysis Method

For all examples GC analysis was performed on an Agilent 7890A GC equipped with an FID and a PolyARC reactor (PolyARC from: Activated Research Company, 7561 Corporate Way, Eden Prairie, MN 55344, USA). The PolyARC reactor converted all analytes to methane before quantification. Helium was used as the carrier and FID makeup gas. Air and H2 were supplied to the PolyARC electronic flow control module and to the FID. The sample was injected without any pretreatment. The effluent of the GC column was sent directly to the inlet of the PolyARC reactor. The reactor effluent was connected directly to the FID. GC conditions: Front inlet Split: 15:1; Inlet temperature: 230 °C; Column: DB-624 (60 m×0.32 mm×1.8 μm); Carrier gas (He): 2 ml/min; Injection volume: 0.5 μL. FID conditions: Temperature: 300 °C; H2:1.5 ml/min; Air: 350 ml/min; Makeup (He): 28 ml/min. PolyARC reactor conditions: Temperature: 450 °C; H2:35 ml/min; Air: 2.5 ml/min. The oven start temperature was 100 °C. After injection, the temperature was raised to 125 °C at 1.5 °C/min, followed by a 5 min hold. The temperature was then raised to 260 °C at 20 °C/min, followed by a 10 min hold. A chromatogram is obtained which is analysed as desired. If nothing else is stated, the quantification is based on peak area.

### Example 2—Biomass Based Composition

A C1-C3 oxygenate mixture was obtained by thermolytic fragmentation of an aqueous sugar (glucose) solution, as disclosed in Example 1 of WO 2017/216311. A hydrogenation product composition was obtained from the C1-C3 oxygenate mixture, as disclosed in Example 4 of U.S. Pat. No. 9,926,247. The hydrogenation product composition thus obtained was condensed to obtain an aqueous solution of ethylene glycol (biomass based composition comprising ethylene glycol).

### Example 3—Distillation

An aqueous solution of ethylene glycol, as provided by Example 2, was distilled to an ethylene glycol concentration of 99.7 wt. % based on the weight of the aqueous solution, as determined by GC-FID analysis (Example 1).

The distillation unit used for the experiment was a continuous distillation unit. It comprised a packed column (column diameter 50 mm; 4 metres of structured packing of Sulzer type DX) with a feed point at the middle of the column. The distillation unit reboiler was a wiped film type heat exchanger. A bottom fraction was collected as the liquid outlet from the wiped film heat exchanger. At the top of the column a water cooled condenser made a total condensation of the vapor from the column. The condensate was split in two according to the reflux ratio, and the reflux fraction was returned as liquid to the top at the column, while the rest was collected as distillate fraction. A liquid side draw was collected through a liquid side draw outlet placed one meter below the top of the column.

The purification by distillation was carried out as three continuous vacuum distillations in series.

In a first distillation step the aqueous solution of ethylene glycol was distilled at a pressure of 200 mBar and at a reflux ratio of 4. Water and byproducts more volatile than propylene glycol were removed as distillate fraction. The bottom fraction was ethylene glycol-enriched.

In a second distillation step the bottom fraction of the first distillation was distilled at a pressure of 200 mBar and at a reflux ratio of 20. Propylene glycol and 1,2-butanediol were completely removed as distillate. The bottom fraction was ethylene glycol-enriched and is the distillation product of the present distillation.

In a third distillation step the bottom fraction of the second distillation was distilled at a pressure of 200 mBar and a reflux ratio of 24. The product stream of the distillation, i.e. a distillate fraction comprising ethylene glycol in an amount of 99.7 wt. %, was collected through the liquid side draw. The product stream composition was the “distillation product”. The distillate to feed mass ratio was 0.13 and the liquid side draw to feed mass ratio was 0.6.

### Example 4—Melt Crystallisation

The melt crystallisation was performed in a cylindrical tank having a cooling jacket. The cooling jacket comprised an inlet and an outlet that were fluidly connected to a source of liquid cooling media. The cylindrical tank comprised an outlet with a valve located at the centre of the bottom of the tank. The inside of the bottom of the tank was covered with a mesh screen, which in use functioned to retain the crystals while the mother liquor was drained through the outlet. The drainage rate was controlled by applying a vacuum on the drain side.

The melt crystallisation involved transferring a sample of the distillation product of Example 3 to the crystallisation jar, and stirring the distillation product using an anchor stirrer at 50 rpm. The stirring prevented the formation of lumps of crystals in the distillation product during the crystallisation. The liquid cooling medium was circulated through the cooling jacket. The temperature of the liquid cooling medium at the inlet of the cooling jacket was set to −22 °C. The distillation product was thereby cooled to a temperature of from around-21 °C to −22 °C. The distillation product was cooled to this temperature without any crystal formation.

To initiate crystallisation, 5 to 10 g of dry ice was crushed to a particle size of 1 to 2 mm and then under stirring poured into the crystallisation jar. The speed of the stirring anchor was then increased to 100 rpm for a few minutes to distribute the dry ice particles in the liquid. Following this, the stirring anchor speed was again reduced to 50 rpm. After a few more minutes, the distillation product became turbid, which indicated that formation of crystals had started. During the crystallisation the circulation of cooling media with a temperature of −22 °C was maintained, while the temperature of the crystal suspension increased to around −15 °C. The crystallisation thus resulted in crystals and a mother liquor in which the crystals were suspended. The mother liquor was removed from the crystallisation jar by application of a vacuum to a bottom valve of the crystallisation jar. Approximately 30 wt. % of the distillation product was removed as mother liquor from the crystallisation jar (based on the weight of the distillation product).

The residue in the crystallization jar after the vacuum filtration was then subjected to two sweating steps, each of which involved partially melting the crystals so as to release impurities trapped with the crystals, by adjusting the temperature of the cooling media to −10 °C.

In a first sweating step, the crystals were partly melted to form a mixture of crystals and a first liquor. The first liquor was then removed by application of a vacuum to the bottom valve of the crystallisation jar. The amount of liquid removed by the vacuum filtration corresponded to around 30 wt. % of the amount of material in the crystallization jar before the sweating was started.

In a second sweating step, the crystals remaining after first sweating step were partly melted to form a mixture of crystals and a second liquor. The second liquor was then removed by application of a vacuum to the bottom valve of the crystallisation jar. The amount of liquid removed by the vacuum filtration corresponded to around 30 wt. % of the amount of material in the crystallization jar before the second sweating step was started.

The crystals obtained from the second sweating step were melted and collected as product. This product is referred to as the “crystallisation product”.

### Example 5—Melt Recrystallization

In order to obtain a further purified product the crystallisation product obtained in Example 4 was subjected to an additional cycle of melt crystallisation and sweating steps as described in Example 3. This further purified product is referred to as the “recrystallisation product”.

### Example 6—Distillation and Melt Crystallisation Product Analysis

The distillation product, the crystallisation product, and the recrystallisation product were evaluated on parameters related to polyester (e.g. polyethylene terephthalate (PET)) production. This involved measurement of: diethylene glycol concentration (wt. %); APHA colour (mg/L PtCo); and APHA colour (mg/L PtCo) after heating to 200 °C for 4 hours.

The standard method for determination of diethylene glycol concentration is described in ASTM E2409-20a.

The standard method for determination of APHA colour is described in the ASTM method D1209-05. The APHA colour measurement (in accordance with ASTM method D1209-05) herein was performed using an instrumental method built into a Lovibond PFX-I Series Spectrocolorimeter. The method is called Pt—Co D1209. The APHA colour was measured using a 100 mm glass cuvette for holding the sample. The measurement was conducted at a temperature of from 20 °C to 25 °C (room temperature) and atmospheric pressure. A standard curve covering the relevant colour range was prepared by volume based dilution of a commercially available Pt—Co standard solution, e.g. available from Sigma Aldrich (Pt—Co/Hazen/APHA Colour Reference Standard, Sigma no. 134190 (ASTM colour 100)), with demineralised water. The APHA value for a given sample was determined from the standard curve based on the measured Pt—Co value for the sample. That is, a given sample was placed in the colorimeter and tested, and the output Pt—Co value from the colorimeter was used against the standard curve to determine the APHA colour value for the given sample.

The heat treatment of the ethylene glycol samples prior to the measurement was achieved by loading each sample into a glass container, and flushing the sample with nitrogen for 15 minutes to remove air. The glass container was sealed, and the container was substantially absent of oxygen. The respective samples in the glass container were heated to 200 °C for four hours. After cooling down, the colour of the heat treated sample was determined using the APHA colour method as outlined above.

Table 1 shows the results of the measurements.

TABLE 1

| | Distillation | Crystallisa- | Recrystallisa- |
|---|---|---|---|
| | Product | tion Product | tion Product |
| Diethylene glycol [wt. %] | <0.05 | <0.05 | <0.05 |
| APHA colour [mg/L PtCo] | 5.6 | 5 | 2.3 |
| APHA colour, after heating | 34.6 | 18.9 | 10.9 |
| [mg/L PtCo] | | | |

The data in Table 1 indicate that the APHA colour of the product was reduced by melt crystallisation and further reduced by subsequent recrystallisation. This applied to both the product before and after the 4 hours of heat treatment. The diethylene glycol content was not negatively impacted by the melt crystallisation steps.

Analysis of the distillation product of Example 3, the crystallisation product of Example 4, and the recrystallisation product of Example 5 by GC analysis method qualitatively demonstrated that the area of all peaks for impurities was reduced by melt crystallisation. Table 2 shows the peak areas for ethylene glycol and three impurities (referred to as impurities 1, 2, and 3); The impurities of peaks 1 to 3 are identified by their retention time in the GC chromatogram. The identity of the impurities was unknown). Table 3 shows the peak areas of impurities 1 to 3 relative to the peak area of ethylene glycol. In Table 4, relative peak areas for the single peaks listed in Table 3 were recalculated as residual impurity content relative to the residual impurity content of the distillation product.

TABLE 2

| | GC Peak area | | | |
|---|---|---|---|---|
| | | Impurity 1 | Impurity 2 | Impurity 3 |
| | Ethylene | (Rt = 15.26 | (Rt = 20.05 | (Rt = 27.6 |
| | glycol | min) | min) | min) |
| Distillation product | 179558 | 122 | 32 | 12 |
| Crystallisation | 180522 | 58 | 9 | 5 |
| product | | | | |
| Recrystallisation | 188102 | 22 | 2 | 2 |
| product | | | | |

TABLE 3

| | Impurity peak area relative to ethylene | | |
|---|---|---|---|
| | glycol peak area *10\hat{ }6 | | |
| | Impurity 1 | Impurity 2 | Impurity 3 |
| | (Rt = 15.26 | (Rt = 20.05 | (Rt = 27.6 |
| | min) | min) | min) |
| Distillation product | 679 | 178 | 67 |
| Crystallisation product | 321 | 50 | 28 |
| Recrystallisation | 117 | 11 | 11 |
| product | | | |

TABLE 4

| | Impurity content relative to distillation product | | |
|---|---|---|---|
| | Impurity 1 | Impurity 2 | Impurity 3 |
| | (Rt = 15.26 | (Rt = 20.05 | (Rt = 27.6 |
| | min) | min) | min) |
| Distillation product | 100 | 100 | 100 |
| Crystallisation product | 47 | 28 | 41 |
| Recrystallisation | 17 | 6 | 16 |
| product | | | |

The data in Table 2 to 4 show that compared to the distilled only product (distillation product) the content of impurities in the purified biomass based composition is reduced by more than 50% by crystallisation and more than 80% by crystallisation followed by recrystallisation. Accordingly, each of the crystallisation product and the recrystallisation product had a greater concentration of ethylene glycol than the distillation product.

### Example 7—Polyester Production

In respective experiments, each of the distillation product of Example 3, the crystallisation product of Example 4, and the recrystallisation product of Example 5 was used to produce a polyester. Each experiment was performed as a standard batch process. Each experiment involved contacting the respective product with terepthalic acid to produce a polyester comprising polyethylene terephthalate (PET). Isophthalic acid (IPA) was added as a copolymerising compound in a 2% fraction over the total diacid compounds. This was performed in two steps: (1) terepthalic acid and the respective product were contacted to result in an esterification reaction, during which bis(2-hydroxyethyl) terephthalate was formed and water and volatile by-products were removed; and (2) bis(2-hydroxyethyl) terephthalate was polymerised (polycondensed) in the presence of an antimony catalyst while released ethylene glycol was continuously removed.

Step (1) was carried out at a temperature in the range of from 150 °C to 260 °C under atmospheric pressure. Step (2) was carried out at a temperature in the range of from 280 °C to 300 °C under vacuum (0.01 to 2 mbar).

Step (2) was performed until the target level of polymerisation was achieved, as determined by measurement of the intrinsic viscosity. The standard method for determination of the intrinsic viscosity is described in ASTM method D4603-18.

The extent of polycondensation can also be approximately determined indirectly by measurement of the torque on the agitator shaft of the mixer in the polymerisation reactor. This requires the preparation of a standard curve for the relation between torque and intrinsic viscosity. This method was used to control the duration of the polycondensation. Once the desired torque on the agitator shaft was reached, the melt of polymerised material was removed from the polymerisation vessel, and transferred to an ice water cooling bath, which stopped the reaction. The value for intrinsic viscosity listed in Table 4, is the actual value measured on the polyester product obtained from the polycondensation. Once cooled, the polymerised material was divided into pellets.

### Example 8—Polyester Product Analysis

The pellets were tested by measurement of intrinsic viscosity; COOH end groups (mmol/kg); and CIELAB colour parameters L*, a*, and b*. The standard method for determination of COOH end group concentration is described in ASTM D7409-15 (at a temperature of from 20 °C to 25 °C and atmospheric pressure).

The standard method for determination of the CIELAB colour parameters is described in ASTM method D6290-19. The method (in accordance with ASTM method D6290-19) herein was conducted at a temperature of from 20 °C to 25 °C and atmospheric pressure. The PET pellets were spread out in a disk (approx. 50 mm in diameter) in an even layer with a thickness of around 10 to 15 mm. The disk was placed in a fully automated colorimeter and the CIELAB colour coordinates were read out on a display.

The main properties of the pellets are listed in Table 5 below.

TABLE 5

| | Distillation | Crystallisation | Recrystallisation |
|---|---|---|---|
| | Product | Product | Product |
| Intrinsic | 0.65 | 0.65 | 0.65 |
| viscosity | | | |
| [g/dl] | | | |
| COOH end | 22 | 18 | 24 |
| groups | | | |
| [mmol/kg] | | | |
| L* | 88.9 | 90.4 | 85.4 |
| a* | −1.6 | −1.4 | −1.2 |
| b* | 4.6 | 1.6 | 0.2 |

As described herein, the L*, a*, and b* parameter values are CIELAB colour components. The L* value expresses perceptual lightness, with black at 0 and white at 100. The a* and b* values express the four colours of human vision: red, green, blue and yellow. Positive b* values are indicative of a yellow colour. The yellow colour decreases as b* approaches 0.

As shown in Table 5, a* was substantially unaffected by the melt crystallisation steps. By contrast, b* was significantly closer to zero as a result of the melt crystallisation steps. The colour of the polyester pellets made from the distillation product was not acceptable for producing bottle grade PET, i.e. PET for production of water and soft drink bottles. The colour of the polyester pellets made from the crystallisation product was acceptable for producing bottle grade PET. The colour of the polyester pellets made from the recrystallisation product was well within the acceptable limits for producing bottle grade PET.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A method of purifying a biomass based composition comprising ethylene glycol, the method comprising:
(a) subjecting the biomass based composition to at least one distillation step to provide a distillation product, wherein the concentration of ethylene glycol in the distillation product is greater than in the biomass based composition; and
(b) subjecting the distillation product to at least one melt crystallisation step to form crystals and a mother liquor, such that the crystals provide a purified biomass based composition, wherein the concentration of ethylene glycol in the purified biomass based composition is greater than in the distillation product.

2. The method according to claim 1, wherein the distillation product comprises ethylene glycol in an amount of no less than 85 wt. %, based on the weight of the distillation product,
optionally wherein the distillation product comprises ethylene glycol in an amount of no greater than 99.95 wt. %, based on the weight of the distillation product.

3. The method according to claim 1, wherein the purified biomass based composition comprises ethylene glycol in an amount of no less than 98 wt. %, based on the weight of the purified biomass based composition.

4. The method according to claim 1, wherein the at least one distillation step comprises a first distillation step comprising feeding the biomass based composition to a continuous distillation unit to provide a first bottom fraction and at least one first distillate fraction wherein one of the first bottom fraction and the at least one first distillate fraction is an ethylene glycol enriched fraction,
optionally wherein the ethylene glycol enriched fraction is the distillation product.

5. The method according to claim 4, wherein the at least one distillation step comprises at least one further distillation step comprising feeding the ethylene glycol enriched fraction from the preceding distillation step to a continuous distillation unit to provide a further bottom fraction and at least one further distillate fraction wherein one of the further bottom fraction and the at least one further distillate fraction is an ethylene glycol enriched fraction,
optionally wherein the ethylene glycol enriched fraction is the distillation product.

6. The method according to claim 1, wherein the biomass based composition to be purified is obtained by thermolytic fragmentation and subsequent hydrogenation of a sugar.

7. The method according to claim 1, wherein step (b) comprises subjecting the distillation product to the at least one melt crystallisation step to form the crystals and the mother liquor, the crystals being present in an amount of at least 50 wt. %, based on the weight of the crystals and the mother liquor.

8. The method according to claim 1, wherein the or each melt crystallisation step comprises heating the crystals to partially melt the crystals to form purified crystals and a residual liquid, wherein the concentration of ethylene glycol in the purified crystals is greater than in the crystals,
optionally wherein from 10 wt. % to 50 wt. % of the crystals are melted to form the residual liquid, based on the weight of the crystals.

9. The method according to claim 1, wherein the at least one distillation step comprises at least partially removing from the biomass based composition one or more of: water, propylene glycol, and 1,2-butanediol.

10. The method according to claim 1, wherein the purified biomass based composition has a UV transmittance at 275 nm determined in accordance with ASTM method E2193-16 of less than 40%.

11. The method according to claim 1, wherein the purified biomass based composition has an APHA colour determined according to ASTM D1209-05 of no greater than 5 mg/L PtCo.

12. The method according to claim 1, wherein the purified biomass based composition has an APHA colour after heating determined according to ASTM D1209-05 of no greater than 20 mg/L PtCo.

13. A purified biomass based composition obtained by the method according to claim 1, the composition having an APHA colour after heating determined according to ASTM D1209-05 of no greater than 20 mg/L PtCo.

14. A method of using a purified biomass based composition obtained by the method according to claim 1 to produce a polyester having one or more of the following CIELAB colour space values determined according to ASTM D6290-19: L* of no less than 65; a* of from −4 to 4; and b* of from −4 to 4.

15. A method of producing a polyester, the method comprising contacting the purified biomass based composition obtained by the method according to claim 1 with at least one reagent to form the polyester having one or more of the following CIELAB colour space values determined according to ASTM D6290-19: L* of no less than 65; a* of from −4 to 4; and b* of from −4 to 4.

16. A polyester obtained by a method according to claim 15, having one or more of the following CIELAB colour space values determined according to ASTM D6290-19: L* of no less than 65; a* of from −4 to 4; and b* of from −4 to 4.

17. The polyester according to claim 16, having one or more of the following CIELAB colour space values determined according to ASTM D6290-19: L* of no less than 85; a* of from −2 to 2; and b* of from −2 to 2.

18. A packaging article or a preform formed from the polyester according to claim 16.

19. The method, the use, a polyester, the packaging article, or the preform according to claim 14, wherein the polyester comprises polyethylene terephthalate.
